(19) United States
(12) Patent Application Publication
(10) Pub. No.: US 2026/0191444 A1
(43) Pub. Date: Jul. 9, 2026

(54) Microtube Cap Having Built-In Compartment for Stabilizer Suited for Remote Blood Collection

(71) Applicant: Blackfly Investments, LLC, dba Molecular Testing Labs, Vancouver, WA (US)

(72) Inventors: Mariko Nakano, Vancouver, WA (US); Ryan Baran-Maguire, Portland, OR (US)

(21) Appl. No.: 19/011,150

(22) Filed: Jan. 6, 2025

(51) Int. Cl. A61B 5/15

(52) U.S. Cl. CPC A61B 5/150351

(57) ABSTRACT

A sealing cap for a specimen tube for use in an automated specimen tube testing system in a laboratory environment. The sealing cap includes a cylindrical aperture having a top end and a bottom end, and an aperture body disposed between the top end and the bottom end, wherein the bottom end comprises a diameter suited to encompass a threaded end of a specimen tube. The sealing cap further includes a compartment defined by at least one mesh side disposed within the cylindrical aperture adjacent to the top end. The mesh side is sized to prevent passing a solid material, such an encompassed blood stabilizer while allowing passing of a liquid material, such as collected blood. In this manner the stabilizer is kept secure and ready to mix with collected blood without risking loss or ingestion in remote collection environments.

### BACKGROUND

As techniques and methodologies for medical testing and diagnostics advance, more consumers are turning to “at-home” collection kits that may involve collecting human material samples, such as blood, urine or saliva, using a collection device without the assistance of a physician/medical staff. In a typical procedure, a patient will induce a blood flow or urine flow into a dedicated collection medium, such as a cup or other receptacle. As is known in the profession, samples collected remotely can then be packaged for mailing or transit to a remote testing facility. At a testing facility, the remotely collected samples may be prepared and tested whereby results of testing or diagnostics may then be communicated to the patient using standard confidentiality protocols.

At the testing facility, the received samples can be processed and prepared using collection and preparation tubes as is standard in the industry. These tubes (e.g., common laboratory test tubes) may have a hard cap that engages the open end of the tube for securing the sample contents inside the tube for in-house maneuvering and storing. When samples are ready for testing and diagnostics, an automated pipette system may be used to dispense test and preparation materials into each tube in an array of tubes with samples. Thus, when a lab technician wishes to work with the sample tubes in the array, the hard cap must be removed (e.g., unscrewed) from each tube. In a system more suited for automation, this human interaction with each tube is time consuming and inefficient.

### BRIEF DESCRIPTION OF THE DRAWINGS

Embodiments of the subject matter disclosed herein in accordance with the present disclosure will be described with reference to the drawings, in which:

FIG. 1 is a plan diagram of a microtube and cap having a mesh compartment for storing a stabilizer suited for remote collection of blood according to an embodiment of the subject matter disclosed herein;

FIG. 2 is a plan view of the cap with stabilizer compartment of FIG. 1 according to an embodiment of the subject matter disclosed herein;

FIG. 3 is a cutaway plan view of the cap with stabilizer compartment of FIG. 1 according to an embodiment of the subject matter disclosed herein;

FIG. 4 is a cutaway isometric view of the cap with stabilizer compartment of FIG. 1 according to an embodiment of the subject matter disclosed herein;

FIG. 5 is a cutaway plan view of a microtube and cap having a mesh compartment for storing a stabilizer suited for remote collection of blood of FIG. 1 according to an embodiment of the subject matter disclosed herein; and

FIG. 6 is system view of an automated pipette system 600 suited for use with an array of specimen tubes 601 each fitted with the sealing cap 140 of FIG. 1 according to an embodiment of the subject matter disclosed herein.

Note that the same numbers are used throughout the disclosure and figures to reference like components and features.

### DETAILED DESCRIPTION

The subject matter of embodiments disclosed herein is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

Embodiments will be described more fully hereinafter with reference to the accompanying drawings, which form a part hereof, and which show, by way of illustration, exemplary embodiments by which the devices described herein may be practiced. These devices may, however, be embodied in many different forms and should not be construed as limited to the embodiments set forth herein; rather, these embodiments are provided so that this disclosure will satisfy the statutory requirements and convey the scope of the subject matter to those skilled in the art.

By way of an overview, the systems and devices discussed herein are directed to a sealing cap for a specimen tube for use in an automated specimen tube testing system in a laboratory environment. The sealing cap includes a cylindrical aperture having a top end and a bottom end, and an aperture body disposed between the top end and the bottom end, wherein the bottom end comprises a diameter suited to encompass a threaded end of a specimen tube. The sealing cap further includes a compartment defined by at least one mesh side disposed within the cylindrical aperture adjacent to the top end. The mesh side is sized to prevent passing a solid material, such an encompassed blood stabilizer while allowing passing of a liquid material, such as collected blood. In this manner the stabilizer is kept secure and ready to mix with collected blood without risking loss or ingestion in remote collection environments.

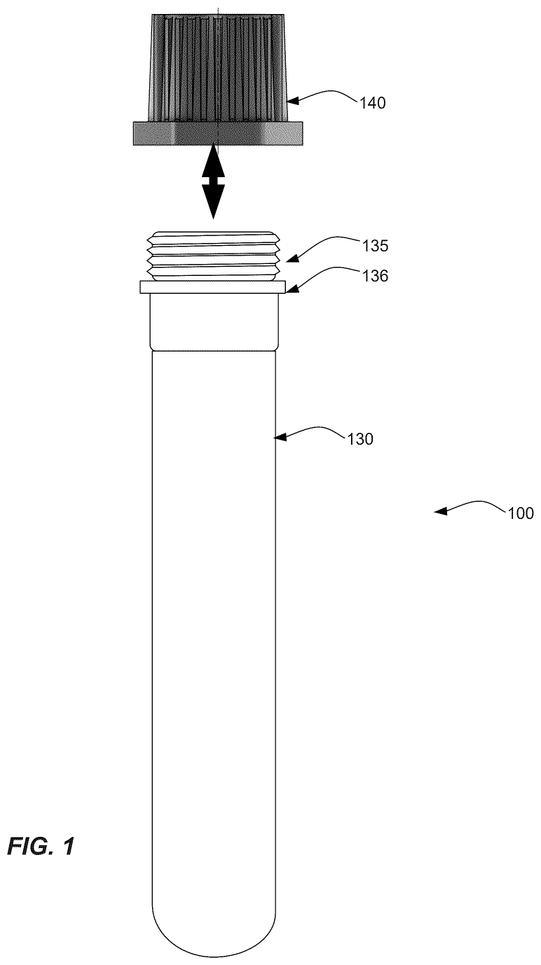

Thus, blood may be collected into a specimen tube at remote locations that may be in need of stabilizers for transport or storage in this convenient manner wherein the stabilizer is stored within the collection specimen tube. Further, the sealing cap may be a pierceable cap for the specimen tube for use in an automated specimen tube testing system in a laboratory environment. In one embodiment, the pierceable cap comprises a cylindrical aperture having a top end, a bottom end and a aperture body disposed between the top end and the bottom end, wherein the bottom end comprises a diameter that is smaller than the top end. This allows the sealing cap to be more easily inserted into a respective specimen tube. The sealing cap further comprises a membrane (typically a foil membrane) disposed over the top end to cover the plugin aperture to form a liquid tight seal, the membrane configured to be pierced by a pipette such that the piercing pipette may pass through the plugin cap aperture. These and other aspects are discussed below with respect to FIGS. 1-6.

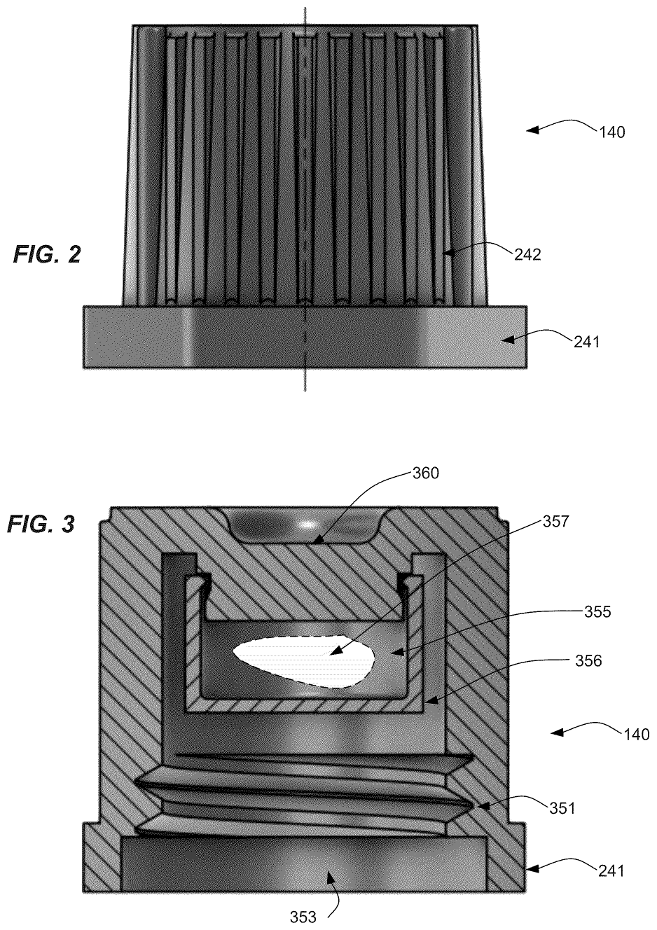

FIG. 1 is a plan diagram of a specimen tube 100 (sometimes called a microtube) pictured adjacent to a cap 130 having a mesh compartment for storing a stabilizer suited for remote collection of blood (unseen if FIG. 1) according to an embodiment of the subject matter disclosed herein. The specimen tube 100 includes a tube body 130 that is an elongated hollow cylinder with a closed end (bottom) and an open end (top) having an aperture for materials and testing equipment access. The aperture end of the specimen tube 100 may include threads 135 disposed around the outer circumference of the specimen tube 100 for engaging a matching sealing cap 140. Thus, the sealing cap 140 may also include reciprocal threads (not shown) inside the inner circumference of the sealing cap 140 to screw the cap down over the aperture of the specimen tube 100. When fully seated (e.g., screwed down tight), the sealing cap seats onto a sealing ledge 136 that is part of the specimen tube 100. The sealing cap 140 is shown in more detail in FIGS. 2-4 described next.

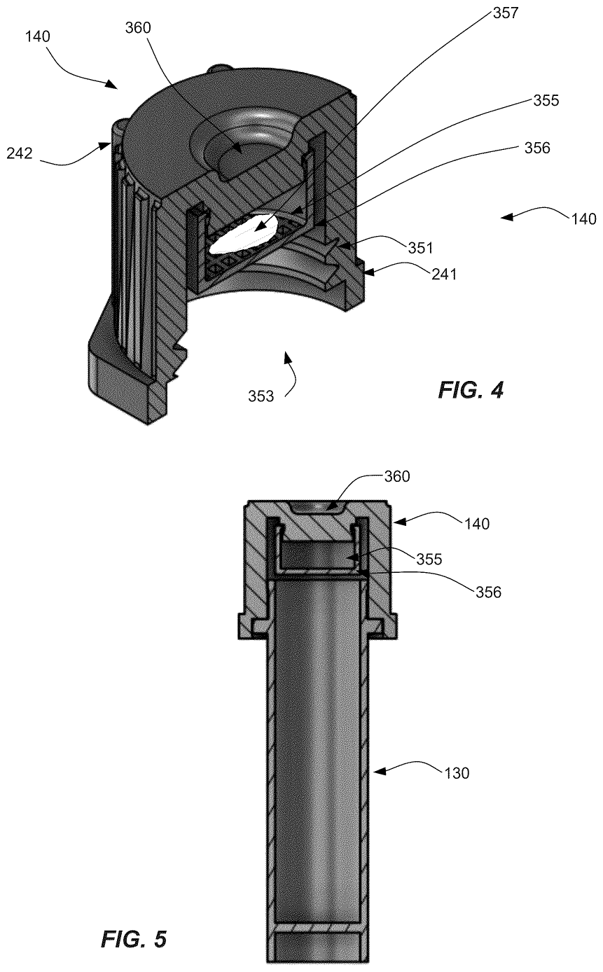

FIG. 2 is a plan view of the sealing cap 140 with stabilizer compartment of FIG. 1 according to an embodiment of the subject matter disclosed herein. As shown in FIG. 2 is greater detail, the sealing cap 140 includes a set of vertically aligned protrusions 242 that enable better purchase when rotating the sealing cap 140 on or off of the specimen tube. As such, when rotated to a sealed position, a sealing ring 242 engages a top surface of the sealing ledge 136 (FIG. 1) of the specimen tube 100. Again, the mesh compartment for storing a stabilizer is unseen in this non-cutaway view of the sealing cap 140. FIGS. 3-4 show cutaway views with detail about the mesh compartment for storing a stabilizer.

FIG. 3 is a cutaway plan view of the sealing cap 140 with stabilizer compartment 355 of FIG. 1 according to an embodiment of the subject matter disclosed herein. In this cutaway view, the sealing cap 140 includes interior threads 351 disposed on the inner circumference of an aperture 353 that are suited to engage with matching threads of a top end of a specimen tube (not shown in FIG. 3). Within this aperture is also a compartment 355 having at least one side (e.g., the bottom side) with a mesh material 356. The mesh material 356 is semi-permeable such that collected blood may pass through the mesh material 356 into the compartment 355 to mix with contents therein. In one embodiment, the mesh compartment 355 stores a solid blood stabilizer 357 to be mixed with collected blood to stabilize for storage and/or transport. This is advantageous because blood stabilizers are harmful if ingested, so securing the stabilizer inside a sealing cap for mixing with collected blood in a specimen tube once sealed by the sealing cap helps prevent accidental loss or ingestion.

The sealing cap 140 further includes a pierceable membrane 360 disposed over the top end to cover the aperture to form a liquid tight seal, the membrane configured to be pierced by a pipette (not shown in FIG. 3) such that the piercing pipette may pass through the aperture. The membrane comprises a foil that may penetrated by a force of approximately 2.4 Newtons and is made from one of foil, plastic, mesh or cloth that do not chemically react with contents of an engaged specimen tube. Further, the overall aperture is approximately 12 mm in diameter suited to encompass an end of a specimen tube.

FIG. 4 is a cutaway isometric view of the cap with stabilizer compartment of FIG. 1 according to an embodiment of the subject matter disclosed herein. In this similar cutaway view, the sealing cap 140 includes interior threads 351 disposed on the inner circumference of an aperture 353 that are suited to engage with matching threads of a top end of a specimen tube (not shown in FIG. 3). Within this aperture is also a compartment 355 having at least one side (e.g., the bottom side) with a mesh material 356. In this embodiment, the cylindrical vertical sides of the mesh compartment 355 is also made of mesh material 356. The mesh material 356 is semi-permeable such that collected blood may pass through the mesh material 356 into the compartment 355 to mix with contents therein. In one embodiment, the mesh compartment 355 stores a solid blood stabilizer 357 to be mixed with collected blood to stabilize for storage and/or transport. For example, once blood is collected in a specimen tube and a sealing cap 140 is placed over the top end of the specimen tube, the entire sealed specimen tube may be inverted such that blood flows toward the mesh compartment having the blood stabilizer. This is advantageous because blood stabilizers are harmful if ingested, so securing the stabilizer inside a sealing cap for mixing with collected blood in a specimen tube once sealed by the sealing cap helps prevent accidental loss or ingestion.

FIG. 5 is a cutaway plan view of a microtube and cap having a mesh compartment for storing a stabilizer suited for remote collection of blood of FIG. 1 according to an embodiment of the subject matter disclosed herein. In this similar cutaway view, the sealing cap 140 includes interior threads 351 disposed on the inner circumference of an aperture 353 that are suited to engage with matching threads of a top end of a specimen tube (not shown in FIG. 3). Within this aperture is also a compartment 355 having at least one side (e.g., the bottom side) with a mesh material 356. In this embodiment, the cylindrical vertical sides of the mesh compartment 355 is also made of mesh material 356. The mesh material 356 is semi-permeable such that collected blood may pass through the mesh material 356 into the compartment 355 to mix with contents therein. In one embodiment, the mesh compartment 355 stores a solid blood stabilizer 357 to be mixed with collected blood to stabilize for storage and/or transport. For example, once blood is collected in a specimen tube 100 and a sealing cap 140 is placed over the top end of the specimen tube 100, the entire sealed specimen tube 100 may be inverted such that blood flows toward the mesh compartment having the blood stabilizer. This is advantageous because blood stabilizers are harmful if ingested, so securing the stabilizer inside a sealing cap for mixing with collected blood in a specimen tube once sealed by the sealing cap helps prevent accidental loss or ingestion.

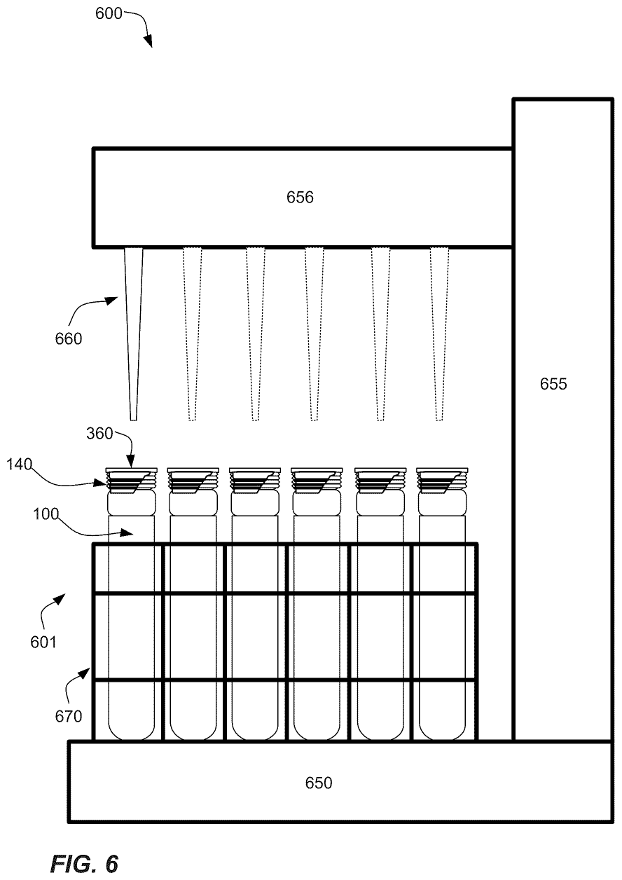

FIG. 6 is system view of an automated pipette system 600 suited for use with an array of specimen tubes 100 each fitted with the sealing cap 140 of FIG. 1 according to an embodiment of the subject matter disclosed herein. The system 600 may include an array of specimen tubes 601 set on a staging platform 650 and held individually upright by a specimen tube holder 670. Each individual specimen tube 100 may contain remotely collected sample materials sealed inside by a respective sealing cap 140 with a respective pierceable membrane 360.

The specimen tube system 600 may also include an automated material dispensing system having at least one pipette configured to penetrate the membrane of a specific sealing cap 140 to deliver material into the corresponding specimen tube 100. In this embodiment, the automated material dispensing system comprises an upright structural member 655 and horizontal pipette actuator 656 that is configured to convey dispensable material to one or more pipettes 660. In this embodiment, each pipette 660 comprises a pointed end having a diameter of no more than 7 mm. In other embodiments, the pointed end of the pipette 660 may have a larger or smaller diameter with the caveat of having to fit inside the aperture of the sealing cap 140. The actuator 656 is configured to maneuver one or more pipettes to align with a respective specimen tube 100 and to lower the one or more pipettes 660 toward each respective specimen tube 100 to pierce the membrane 360 of each respective sealing cap 140. Once the pipette 660 is maneuvered inside the specimen tube 100, the media therein may be aspirated and/or additional materials (such as reagents) may be dispensed into the specimen tube 100. The actuator 656 may then retract the pipette 660 out of the specimen tube 100.

The specimen tube holder 670 may hold a plurality of specimen tubes 100 and is arranged in an array of bins wherein each bin is configured to hold one of the plurality of specimen tubes 100 upright such that a pipette 660 may be maneuvered into each upright specimen tube 100 by penetrating a respective sealing cap membrane 360 for delivery of materials through the pipette 660. In one embodiment, the holder 670 comprises and array of six by six.

Utilizing the system 600 of FIG. 6 enables a technician to perform a method for testing a specimen held in a specimen tube 100. In an embodiment, the method comprises collecting a specimen from a human in a remote location (e.g., away from a laboratory setting such as at home) and then receiving the remotely collected yet stabilized specimen at a local laboratory. The technician may then prepare an elution using the received specimen in a specimen tube 100. Once the specimen tube 100 is sealed and stabilized, the technician may continue by placing the sealed specimen tube 100 in a testing system array (e.g., holder 670) and maneuvering a pipette 660 to the specimen tube 100 and piercing the membrane 360 with the pipette 660 such that the pipette 660 enters the specimen tube 100 while the membrane 360 remains adhered to an aperture of the specimen tube 100. When pierced, the technician may enable dispensing material into the specimen tube 100 and retracting the pipette 660 from specimen tube 430. One or more of these steps may be automated without enablement or initiation by the technician.

The use of the terms “a” and “an” and “the” and similar referents in the specification and in the following claims are to be construed to cover both the singular and the plural, unless otherwise indicated herein or clearly contradicted by context. The terms “having,” “including,” “containing” and similar referents in the specification and in the following claims are to be construed as open-ended terms (e.g., meaning “including, but not limited to,”) unless otherwise noted. Recitation of ranges of values herein are merely indented to serve as a shorthand method of referring individually to each separate value inclusively falling within the range, unless otherwise indicated herein, and each separate value is incorporated into the specification as if it were individually recited herein. All methods described herein can be performed in any suitable order unless otherwise indicated herein or clearly contradicted by context. The use of any and all examples, or exemplary language (e.g., “such as”) provided herein, is intended merely to better illuminate embodiments and does not pose a limitation to the scope of the disclosure unless otherwise claimed. No language in the specification should be construed as indicating any non-claimed element as essential to each embodiment of the present disclosure.

Different arrangements of the components depicted in the drawings or described above, as well as components and steps not shown or described are possible. Similarly, some features and sub-combinations are useful and may be employed without reference to other features and sub-combinations. Embodiments have been described for illustrative and not restrictive purposes, and alternative embodiments will become apparent to readers of this patent. Accordingly, the present subject matter is not limited to the embodiments described above or depicted in the drawings, and various embodiments and modifications can be made without departing from the scope of the claims below.

That which is claimed is:

1. A sealing cap for a specimen tube, comprising:
a cylindrical aperture having a top end and a bottom end, and an aperture body disposed between the top end and the bottom end, wherein the bottom end comprises a diameter suited to encompass a threaded end of a specimen tube; and
a compartment defined by at least one mesh side disposed within the cylindrical aperture adjacent to the top end, the at least one mesh side sized to prevent passing a solid material while allowing passing of a liquid material.

2. The sealing cap of claim 1, wherein the liquid material suited to pass through the mesh side comprises human blood.

3. The sealing cap of claim 1, wherein the solid material preventing from passing through the mesh side comprises a human blood stabilizer.

4. The sealing cap of claim 1, wherein the compartment further comprises a cylindrical shape.

5. The sealing cap of claim 1, further comprising threads disposed on an inside circumference of the aperture configured to engage the threaded end of a specimen tube.

6. The sealing cap of claim 1, further comprising a pierceable membrane disposed over the top end to cover the aperture to form a liquid tight seal, the membrane configured to be pierced by a pipette such that the piercing pipette may pass through the aperture.

7. The sealing cap of claim 6, wherein the membrane comprises a foil that may penetrated by a force of approximately 2.4 Newtons.

8. The sealing cap of claim 6, wherein aperture is approximately 12 mm in diameter.

9. The sealing cap of claim 6, wherein membrane comprises one from the group composed of foil, plastic, mesh and cloth.

10. The sealing cap of claim 6, wherein membrane comprises one or more materials that do not chemically react with contents of an engaged specimen tube.

11. A specimen tube system, comprising:
a specimen tube having a cylindrical body with a specimen tube aperture on a tube end exposing a holding area inside the cylinder; and
a sealing cap engageable with the specimen tube, having:
a cylindrical aperture having a top end and a bottom end, and an aperture body disposed between the top end and the bottom end, wherein the bottom end comprises a diameter suited to engage the specimen tube;
a compartment defined by at least one mesh side disposed within the cylindrical aperture adjacent to the top end, the at least one mesh side sized to prevent passing a solid material while allowing passing of a liquid material; and
a solid blood stabilizer disposed in the compartment sized to be prevented from passing through the at least one mesh side.

12. The specimen tube system of claim 11, further comprising:
specimen tube threads disposed on an outer circumference of the specimen tube adjacent to the specimen tube aperture; and
sealing cap threads disposed on an inside circumference of the sealing cap aperture configured to engage the threads of the specimen tube in a liquid-tight sealed manner.

13. The specimen tube system of claim 11, further comprising a membrane disposed over the top end of the sealing cap, the membrane configured to be pierced by a pipette such that the piercing pipette may pass through the sealing cap aperture.

14. The specimen tube system of claim 11, further comprising:
an automated material dispensing system having at least one pipette configured to penetrate the membrane of the pierceable plugin cap to deliver material into the specimen tube, the pipette comprising a pointed end having a diameter of no more than 7 mm.

15. The specimen tube system of claim 11, further comprising:
at least one pipette configured to dispense material into specimen tubes; and
an actuator configured to maneuver the pipette in and out of the specimen tube.

16. A method for collecting blood in a specimen tube, the method comprising:
collecting blood from a human in a specimen tube at a location remote from a laboratory;
placing a sealing cap over a top end of the specimen tube, the sealing cap having a compartment defined by at least one mesh side disposed within the sealing cap and holding a blood stabilizer in solid form;
inverting the sealed specimen tube to cause the collected blood to permeate the compartment and mix with the stabilizer stored therein; and
packaging and sending the specimen tube having the collected blood mixed with the stabilizer to the laboratory.

17. The method of claim 16, further comprising:
placing the sealed specimen tube in a testing system array;
maneuvering a pipette to the specimen tube and piercing a membrane disposed on a top side of the sealing cap with the pipette such that the pipette enters the specimen tube while the membrane remains adhered to the aperture of the sealing cap;
dispensing material into the specimen tube; and
retracting the pipette from specimen tube such that materials in the specimen tube remain in the specimen tube.

18. The method of claim 16, further comprising testing the collected blood for presence of an enzyme.

19. The method of claim 16, further comprising testing the collected blood for presence of an infection.

20. The method of claim 16, further comprising testing the collected blood for presence of contamination.
